(19) United States
(12) Patent Application Publication
(10) Pub. No.: US 2012/0179121 A1
(43) Pub. Date: Jul. 12, 2012

(54) Dial-Cap

(76) Inventor: James E. Carson, Colorado Springs, CO (US)

(21) Appl. No.: 13/290,277

(22) Filed: Nov. 7, 2011

Related U.S. Application Data
(60) Provisional application No. 61/430,199, filed on Jan. 6, 2011.

(51) Int. Cl. A61M 35/00

(52) U.S. Cl. 604/290; 604/307

(57) ABSTRACT

An architecture is presented that provides a topical cream delivery device designed for effectively and easily administering hydrating and pain-relieving creams to dialysis patients' and other “in need” patients' needle sites. The topical cream delivery device comprises an adhesive bandage strip and a cavity positioned along the adhesive bandage strip. The cavity is typically filled with a hydrating and pain-relieving cream for dialysis patients, but could be filled with any other suitable cream or drug as well. The adhesive bandage strip further comprises an adhesive backing, for adhering to a user's skin. The adhesive bandage strip is attached to the adhesive backing on one side and covered with a protective paper on the other and cut to shape for application to a body surface. A user when using the topical cream delivery device peels off the protective paper from the adhesive backing and adheres the device to a body surface.

### CROSS-REFERENCE

This application claims priority from Provisional Patent Application Serial No. 61/430,199 filed Jan. 6, 2011.

### BACKGROUND

Dialysis patients typically apply various creams to needle sites where dialysis treatments will take place, as well as do other patients who are frequently required to be injected with medications or have blood drawn on a frequent basis. These creams can be hydrating creams that moisturize the skin and/or pain-relieving creams that lessen the pain associated with dialysis treatments and other types of injections or treatments. Generally, doctors recommend applying the cream one hour before dialysis. After the cream is applied to the arm or leg (dialysis requires two needle sticks), it is then wrapped with plastic wrap to try to keep the cream close to the needle sites and limit contact with clothing. This can be a difficult and arduous task, as often times the cream can ooze out of the sides of the plastic wrap. Additionally, when pressure is applied to the plastic wrap, the cream can become displaced and will no longer be directly over the needle site, thereby reducing the effectiveness of the cream. An effective solution is necessary.

There is a need for a topical cream delivery device that effectively and easily administers hydrating and pain-relieving creams to a dialysis patient's needle sites. The present invention discloses a sterile, dome-shaped elongated bandage that allows dialysis patients to administer the appropriate amount of cream to their needle site areas. The dialysis patient can simply fill the dome with the cream, line up the dome with the needle site area, and apply the bandage to the skin. After the needle site is saturated, the patient can simply remove and dispose of the bandage. Due to the easy-to-use design of the bandage, dialysis patients as well as all other qualified patients will be able to use this convenient product with minimal effort while benefitting from maximized results. Qualified patients could be persons requiring pre-surgical IVs, chemotherapy, injections, or repetitive shots in a concentrated area of the skin. Patients with burn areas that need treating, as well as patients needing post surgical scar reduction therapy could also benefit from this product.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a topical cream delivery device designed for effectively and easily administering hydrating and pain-relieving creams to dialysis patients' needle sites. The topical cream delivery device comprises an adhesive bandage strip and a generally dome-shaped cavity positioned on the adhesive bandage strip. The cavity is typically filled with a hydrating and pain-relieving cream for dialysis patients, but could be filled with any other suitable cream or drug as well.

Furthermore in the preferred embodiment of the present invention, the adhesive bandage strip further comprises an adhesive backing for adhering to a user's skin. The adhesive bandage strip can be attached to an adhesive backing on one side and covered with a protective paper on the other and cut to shape for application to a body surface. A user when using the topical cream delivery device would peel off the protective paper from the adhesive backing and adhere the device to the desired body surface.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of a topical cream delivery device in accordance with the disclosed architecture.

FIG. 2 illustrates a perspective view of the topical cream delivery device wherein the adhesive backing is removed in accordance with the disclosed architecture.

FIG. 3 illustrates a perspective view of the cavity of the topical cream delivery device being filled with cream in accordance with the disclosed architecture.

FIG. 4A and B illustrates a perspective view of the topical cream delivery device in two sizes in accordance with the disclosed architecture.

FIG. 5 illustrates the topical cream delivery device in use in accordance with the disclosed architecture.

FIG. 6A-D illustrates a user utilizing the topical cream delivery device in accordance with the disclosed architecture.

### DESCRIPTION OF PREFERRED EMBODIMENTS

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a topical cream delivery device that effectively and easily administers hydrating and pain-relieving creams or other medications to a dialysis patient's needle sites. The topical cream delivery device discloses a sterile, generally dome-shaped bandage that allows dialysis patients to administer the appropriate amount of cream to their needle site areas. The dialysis patient can simply fill the dome with the appropriate type and amount of cream and/or medication, line up the dome with the needle site area, and apply the bandage to the skin. After the needle site is saturated, the patient can simply remove and dispose of the delivery device.

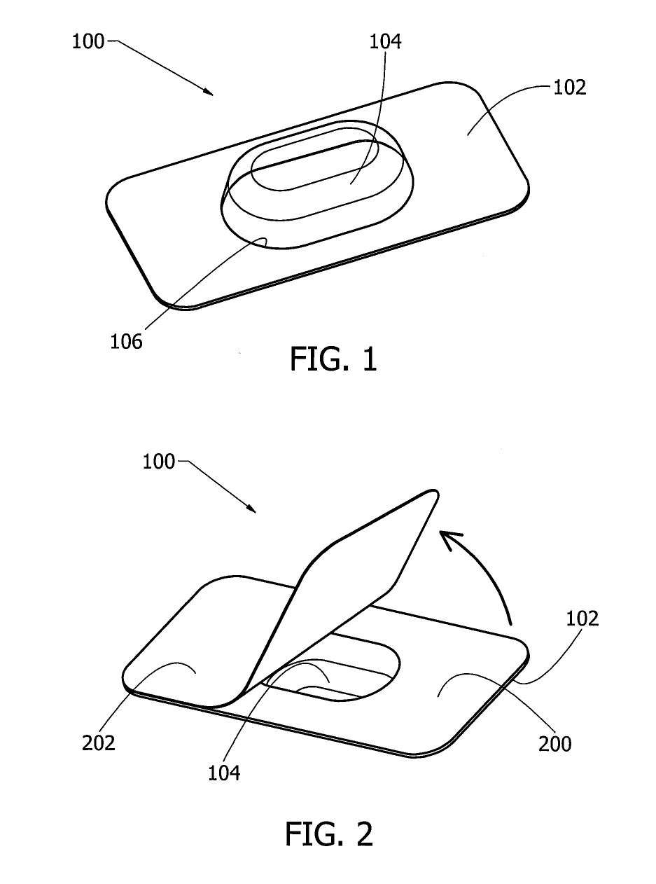

Referring initially to the drawings, FIG. 1 illustrates a topical cream delivery device or topical dressing 100 designed for effectively and easily administering hydrating and pain-relieving creams and other medications to dialysis patients' needle sites. The topical cream delivery device 100 comprises an adhesive bandage strip 102 and a cavity 104 positioned along the adhesive bandage strip 102. The bandage strip 102 is typically a rectangular shaped bandage strip, but can be any suitable shape or size as is known in the art without affecting the overall concept of the invention, such as oval, square, circular, etc. Furthermore, the bandage strip 102 is any typical bandage strip as is known in the art and is generally comprised of a composition that uses a blend of polyisobutylene, polybutene, butyl rubber with reinforcing fiber, filler material and zinc oxide blended under heat and rolled into a thin sheet, though any other suitable material may be used to manufacture the bandage strip 102 as is known in the art without affecting the overall concept of the invention.

The cavity or receptacle 104 is positioned on the adhesive bandage strip 102 and is typically dome-like in shape, but can be any suitable shape or size as is known in the art without affecting the overall concept of the invention provided that the same is capable of delivering the cream and/or medication to the desired site in the manner described herein. The cavity 104 is approximately between ½ and ¾ inches wide, and approximately between ¼ and ⅜ inches high, and approximately between ¾ and 3 inches long. Furthermore, the cavity 104 comprises a rim 106 that is pressed into the bandage strip 102. The rim 106 helps to secure the cavity 104 to the bandage strip 102. Specifically, the rim 106 of the cavity 104 will be pressed into a pre-cut hole in the bandage strip 102 to secure the cavity 104 to the bandage strip 102. However, any other suitable method of securing the cavity 104 to the bandage strip 102 can be used without affecting the overall concept of the invention.

FIG. 2 illustrates the topical cream delivery device 100 further comprising an adhesive backing 200 for adhering to a user's skin. The topical cream delivery device 100 comprises an adhesive bandage strip 102 and a cavity 104 positioned on the adhesive bandage strip 102. Furthermore, the adhesive bandage strip 102 can be attached to an adhesive backing 200 on one side and covered with a protective paper 202 on the other and cut to shape for application to a body surface. A user when using the topical cream delivery device 100 would peel off the protective paper 202 from the adhesive backing 200 and adhere the device 100 to the desired body surface. Generally, doctors recommend applying the cream one hour before dialysis.

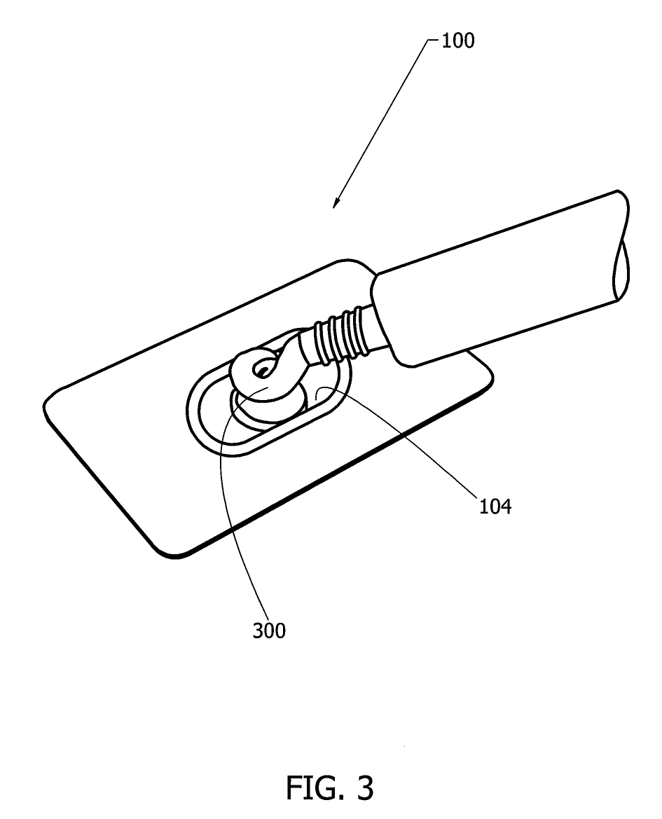

FIG. 3 illustrates the topical cream delivery device 100 wherein the protective paper is removed, allowing access to the cavity 104. The cavity 104 is typically filled with a hydrating and pain-relieving cream 300 for dialysis patients, however any other suitable cream or drug can be used without affecting the overall concept of the invention. For example, a time release drug can be used to administer a predetermined amount of medicine to a user over an extended time period. A user fills the cavity 104 with the cream 300 as shown in FIG. 3, then lines up the cavity 104 with the needle site and applies the delivery device 100 to the user's skin. After the needle site is saturated, the user can simply remove and dispose of the delivery device 100.

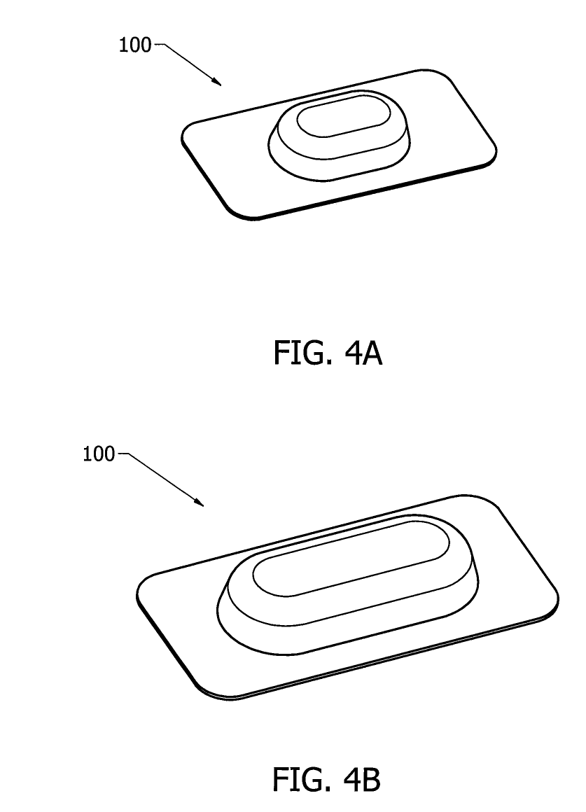

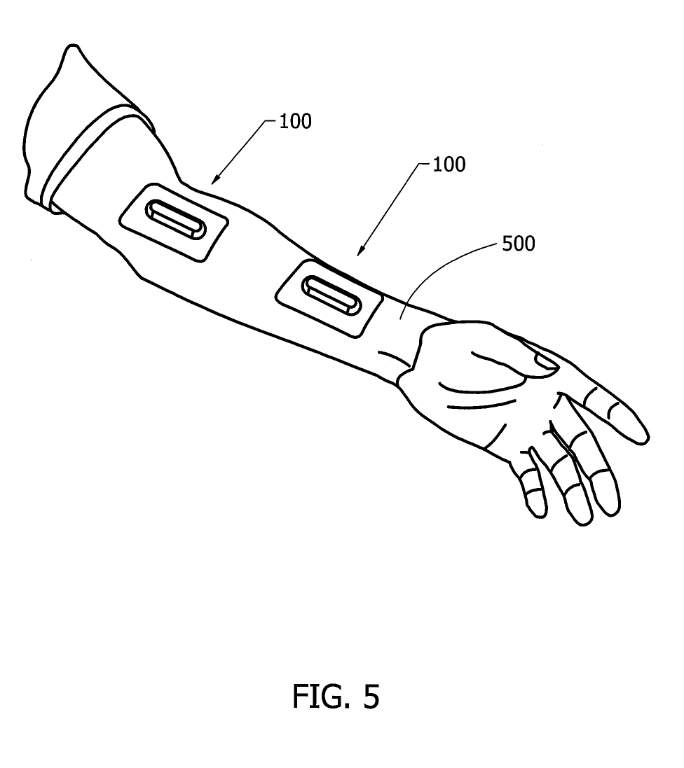

FIG. 4A and B illustrates the topical cream delivery device 100 in two sizes. The topical cream delivery device 100 can be manufactured in a variety of sizes depending on the wants and needs of a user without affecting the overall concept of the invention. Typically, the delivery device 100 can be manufactured in two sizes, regular (see FIG. 4A) which measures approximately 2½ inches long by 2 inches wide, and large (see FIG. 4B) which measures approximately 3½ inches long by 2 inches wide.

FIG. 5 illustrates the topical cream delivery device 100 in use. As shown, the protective paper of the adhesive backing is removed, cream is applied to the cavity, and the topical cream delivery device 100 is adhered to a user's body surface 500. Once the needle site is saturated, the delivery device 100 can be removed and disposed of. Thus, the topical cream delivery device 100 allows dialysis patients to effectively and easily administer hydrating and pain-relieving creams to their needle sites. Due to the easy-to-use design of the delivery device, dialysis patients will be able to use the delivery device with minimal effort while benefitting from maximized results.

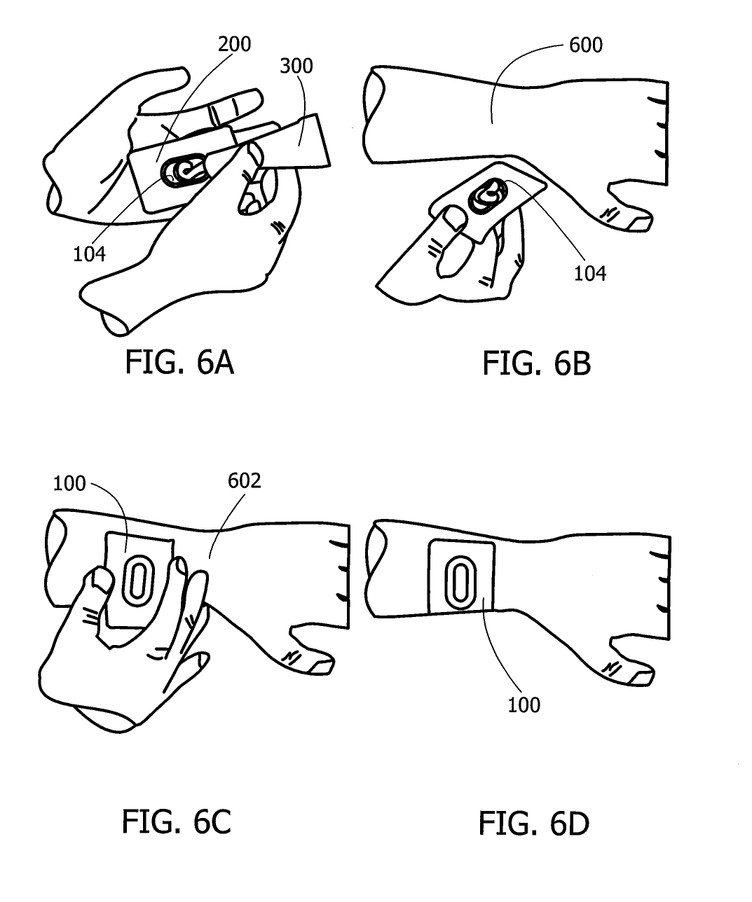

Furthermore, FIG. 6A-D illustrates a user utilizing the topical cream delivery device 100. As shown in FIG. 6A, a user takes a delivery device 100 out of a storage receptacle (not shown), and peels off the protective paper (not shown), exposing the adhesive backing 200 and the cavity 104. A user can then fill the cavity 104 with a predetermined amount of hydrating and pain-relieving cream 300. As shown in FIG. 6B, a user then lines-up the cavity 104 with the needle site 600. As shown in FIG. 6C, a user applies the delivery device 100 to the user's skin 602. As shown in FIG. 6D, the delivery device 100 is secured in place over the needle site (not shown) for approximately 1 hour before dialysis, till the needle site is saturated. After the needle site is saturated, the user can simply remove and dispose of the delivery device 100. Thus, the delivery device 100 facilitates comfort and ease while reducing mess and clothing stains.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A topical cream delivery device, comprising:
an adhesive bandage strip; and
a cavity positioned along the adhesive bandage strip.

2. The device of claim 1, wherein the cavity is generally dome-shaped.

3. The device of claim 1, wherein the cavity is filled with a cream.

4. The device of claim 3, wherein the cream is a hydrating and pain-relieving cream for dialysis needle sites.

5. The device of claim 1, wherein the cavity is filled with a drug.

6. The device of claim 1, wherein the bandage strip has an adhesive backing for adhering to a user's skin.

7. The device of claim 1, wherein the delivery device is manufactured in different sizes depending on a user's need.

8. The device of claim 2, wherein the cavity further comprises a rim.

9. The device of claim 8, wherein the rim is attached to the adhesive bandage strip.

10. A topical dressing, comprising:
an adhesive bandage strip; and
a generally dome-shaped cavity positioned along the adhesive bandage strip; and
wherein the dome-shaped cavity comprises a cream.

11. The dressing of claim 10, wherein the adhesive bandage strip has an adhesive backing for adhering to a user's skin.

12. The dressing of claim 10, wherein the dome-shaped cavity further comprises a rim.

13. The dressing of claim 12, wherein the rim is attached to the adhesive bandage strip to secure the cavity to the adhesive bandage strip.

14. The dressing of claim 10, wherein the dressing is manufactured in different sizes depending on a user's need.

15. The dressing of claim 10, wherein the cream is a hydrating and pain-relieving cream for dialysis needle sites.

16. A method of delivering a topical cream to a dialysis needle site, comprising:
removing a topical cream delivery device from a storage container, the topical cream delivery device comprising an adhesive bandage and a cavity positioned along the adhesive bandage;
filling the cavity with a predetermined amount of cream; and
placing the delivery device over a needle site.

17. The method of claim 16, wherein the cream is a hydrating and pain-relieving cream for dialysis needle sites.

18. The method of claim 16, further comprising peeling off a protective paper to expose an adhesive backing and the cavity.

19. The method of claim 16, further comprising saturating the needle site with the cream.

20. The method of claim 16, further comprising:
removing the topical delivery device after a predetermined amount of time; and
disposing of the topical delivery device.
